(19) United States
(12) Patent Application Publication
(10) Pub. No.: US 2007/0122375 A1
(43) Pub. Date: May 31, 2007

(54) Bile acid sequestrant and process for preparation thereof

(75) Inventors: Mahesh Ambadas Gore, Maharashtra (IN); Mohan Gopal Krishna Kulkarni, Maharashtra (IN)

Correspondence Address: LADAS & PARRY, 26 WEST 61ST STREET, NEW YORK, NY, 10023, US

(73) Assignee: COUNCIL OF SCIENTIFIC AND INDUSTRIAL RESEARCH

(21) Appl. No.: 11/519,999

(22) Filed: Sep. 12, 2006

(30) Foreign Application Priority Data
Sep. 12, 2005 (IN) 2461/DEL/2005

(51) Int. Cl. A61K 31/787; C08G 63/91

(52) U.S. Cl. 424/78.3; 525/54.2

(57) ABSTRACT

The present invention describes a copolymer comprising multiple unsaturations, which are obtained by polymerization of dimethyl β-cyclodextrin inclusion complex of monomer containing multiple vinyl unsaturation and amine functional monomer. These water soluble copolymers containing unsaturation sites, crosslinked in the presence of bile acid template. This novel sequential polymerization and crosslinked process enhances the rebinding capacity of the bile acid sequestrant for the bile acid used as the template during crosslinking step and also selectivity over other bile acids, in comparison to the polymers synthesized by the conventional simultaneous polymerization/crosslinking method.

### FIELD OF THE INVENTION

The present invention relates to a novel bile acid sequestrant [BAS] and a process for preparation thereof. More particularly, the present invention provides water soluble copolymers containing unsaturation sites, crosslinked in the presence of bile acid template. This novel sequential polymerization and crosslinking process enhances rebinding capacity of the bile acid sequestrant for bile acid used as the template during crosslinking step and also selectivity over other bile acids, in comparison to the polymers synthesized by conventional simultaneous polymerization/crosslinking methods.

### BACKGROUND OF THE INVENTION

Clinical evidence has demonstrated a relationship between increased blood cholesterol level and an increased risk of coronary heart disease. When the total cholesterol synthesized and obtained from a person's diet exceeds the amount necessary for synthesis of membrane bile acids and steroids, it accumulates in blood vessels and develops atherosclerotic plaque. Increase in cholesterol levels results in atherosclerosis.

The BAS acts as anion exchange resin, binding bile acid in the lumen of the small intestine. BAS interrupts the enterohepatic circulation of bile acids. This results in increased hepatic synthesis of bile acids from cholesterol. Some of this cholesterol is derived from plasma, which results in the net reduction of plasma cholesterol [J. E. Polli, G. L. Amidon, J. Pharm. Sci., 84, [1995], 55,].

A wide range of BAS have been exploited for reduction of hypercholesterolemia [E. R. Stedronsky, Biochim. Biophys. Acta, 210, [1994], 255, W. H. Mandeville, D. I. Goldberg, Curr. Pharm. Res., 3, [1997], 15, G. M. Benson, D. R. Alston, D. M. B. Hickey, A. A. Jaxa-Chamiec, C. M. Whittaker, C. Haynes, A. Glen, S. Blanchard, S. R. Cresswell, J. Pharm. Sci., 86, [1997], 76. Mandeville and Holmes-Farley [W. H. Mandeville and S. R. Holmes-Farley U.S. Pat. No. 6,433,026 [2002], U.S. Pat. No. 5,607,669 [1997], U.S. Pat. No. 5,679,717 [1997], U.S. Pat. No. 5,693,675 [1997], U.S. Pat. No. 5,917,007 [1999], U.S. Pat. No. 5,919,832 [1999], U.S. Pat. No. 6,066,678 [2000]] synthesized primary as well as quaternary BAS and demonstrated a method for binding bile salts in a mammal. Other anion exchange resins based on copolymers of styrene, divinylbenzene and quaternized with various functional groups such as trimethylamine, imidazoles, pyridinium groups etc. have been prepared and investigated for their potential use as bile acid sequestering agents [D. A. Cook, J. C. Godfrey, D. L. Schneider, J. (Rubinfeld, DE28225467, [1978], A. F. Wagner, U.S. Pat. No. 2,806,707, [1978], A. A. Jaxa-Chamiec, D. M. B. Hickey, P. V. Shah, EP402062, [1990]].

Anion-exchange resins based on acrylic monomers have been prepared and investigated for treatment of hyperlipoproteinemia [V. Borzatta, M. Cristofori, A. Brazzil, EP12804, [1980], N. Grier, T. Y. Shel, A. P. Wagner, U.S. Pat. No. 4,205,064, [1980], L. E. St-Pierre, G. R Brown, D. S. Herding, M. Bouvier, U.S. Pat. No. 4,593,073, [1986], X. X. Zhu, G. R. Brown, L. E. St-Pierre, J. Macromol. Pure Appl. Chem., 29, [1992], 711, K. Kobayashi, O. Hirata, JP07126175, [1995]]. A common limitation of these resins is large dose requirement because of their lower capacity as well as selectivity, especially under in-vivo conditions. Enhanced binding capacity and selectivity is a desirable feature of bile acid sequestrants.

Molecular imprinting technique involves pre-organization of functional monomers around a template molecule, which resembles shape and size of the guest molecule, by either covalent, non-covalent or co-ordination interactions. Polymerization of the supramolecular assembly in the presence of an excess of crosslinker and subsequent removal of the template leads to polymers that retain the specific orientation of functional groups within the cavity created by the elution of the template molecule [G. Wulff, Angew. Chem. Int. Ed. Engl., 34, [1995], 1812, K. J Shea, Trends Polym. Sci., 2, [1994], 166, A. G. Mayes, K. Mosbach,] One of the limitations of the molecularly imprinted polymers prepared by this method is their low rebinding capacity. Enhancing binding capacity of the molecularly imprinted polymers is desirable.

Recovery of bile acids using molecular imprinting technique is known [C. C. Huval, J. B. Mathew, H. B. William, S. H. Randall, W. H. Mandeville, J. S. Petersen, S. C. Polomoscanik, R. J. Sacchiro, Xi Chen, and P. K. Dhal, Macromolecules, 34, [2001] 1548]. Molecularly Imprinted polymers were synthesized by partially neutralizing poly [allylamine hydrochloride] and crosslinking with epichlorohydrin in presence of the template sodium cholate [NaC].

### OBJECTS OF THE INVENTION

The main object of the present invention is to provide novel bile acid sequestrants.

It is another object of the invention to provide a process for preparation of copolymers of dimethyl β-cyclodextrin inclusion complex of crosslinker and a functional monomer.

Yet another object is to provide a crosslinked copolymer, crosslinked in the presence of NaC or sodium taurocholate [NaT] template in an aqueous medium.

Yet another object is to provide a process for the preparation of bile acid sequestrants, which has a higher capacity and selectivity for the rebinding of NaC or NaT by extracting the template molecule from the crosslinked copolymer.

### SUMMARY OF THE INVENTION

The present invention describes copolymers comprising multiple unsaturations, that are obtained by polymerization of dimethyl β-cyclodextrin inclusion complex of monomer containing multiple vinyl unsaturation and functional monomer. The comonomer, which can be used in synthesis of bile acid sequestrants is selected from 2-[methyl [acryloyl oxyethyl] trimethyl ammonium chloride, N-acryloyl-6-amino caproyl hydrochloride, N-acryloyl 5-amino caproyl hydrochloride, 2-amino ethyl acrylate, 2-amino ethyl methacrylate hydrochloride, vinyl amine hydrochloride or allylamine hydrochloride. The monomers containing multiple unsaturations, which can be used in the synthesis of these polymers, are exemplified by methylene bisacrylamide [MBAM] and ethylene bis methacrylamide [EBMA].

The invention also provides a process for preparation of copolymers of a crosslinker and a functional monomer for synthesis of bile acid sequestrants. This invention describes copolymerization of dimethyl β-cyclodextrin complex of a crosslinker such as MBAM or EBMA with allylamine hydrochloride or 2-amino ethyl methacrylate hydrochloride. One of the vinyl groups remains unpolymerized and a water soluble copolymer is obtained. In the second stage, this copolymer is crosslinked in the presence of NaC or sodium taurocholate [NaT] template in aqueous medium. The template molecule is extracted and resulting polymer tested for rebinding of NaC or NaT from phosphate buffer [pH 7.4]. Results show higher percentage utilization [70-84%] of active site as well as selectivity for the rebinding of NaC and NaT.

Water insoluble molecules become water soluble on treatment with aqueous solutions of cyclodextrin or its derivatives. The inclusion phenomenon leads to significant changes in reactivity and solution properties of the guest molecule. The formation of inclusion complexes of hydrophobic monomers with β-cyclodextrin or its derivatives has been reported. [J. Storsberg, H. Ritter, Macromolecular Rapid Communications, 21, [2000], 230, J. Jeromin, H. Ritter, Macromolecular Rapid Communications, 19, [1998], 377, J. Jeromin, O. Noll, H. Ritter, Macromolecular Chemistry & Physics, 199, [1998], 2641, P. Glockner, H. Ritter, Macromolecular Rapid Communications, 20, [1999], 602].

The formation of inclusion complex leads to solubilization of hydrophobic compounds in aqueous media [G. Wenz, Angew. Chem., 106, [1994], 851]. The use of cyclodextrin to dissolve hydrophobic monomers in water has been described in the literature [J. Storsberg, H. Ritter, Macromolecular Rapid Communications, 21, [2000], 236, J. Jeromin, H. Ritter, Macromolecular Rapid Communications, 19, [1998], 377, J. Jeromin, O. Noll, H. Ritter, Macromolecular Chemistry & Physics, 199, [1998], 2641, P. Glockner, H. Ritter, Macromolecular Rapid Communications, 20, [1999], 602]. Some patents describe the use of cyclodextrin preferably in catalytic amounts in order to improve emulsion polymerization yields [G. Siegfried, O. Michael, D. Michael, G. Thomas, L. Christoph, U.S. Pat. No. 6,225,299, [2001] and L. Willie, U.S. Pat. No. 5,521,266, [1996]]. A process for preparation of inclusion complexes of cyclic macromolecular compounds with monomers containing multiple unsaturations is reported in our co-pending applications US 2005032995, WO2005014671 & PCT/IB03/05070]. Polymerization of such complexes with vinyl substituted monomers yields polymers that are soluble and have unsaturated sites for further modification.

### DETAILED DESCRIPTION OF THE INVENTION

The present invention relates to a bile acid imprinted crosslinked polymer having the formula, [A_{[x]} B_{[y]}]ₙ where A is an amine containing monomer having single unsaturation, B is a vinyl monomer having multiple unsaturation, x=1 to 15, y=1 to 15 and n=10 to 1000.

The present invention also provides a process for preparation of bile acid imprinted crosslinked polymer having general formula [A_{[x]} B_{[y]}]ₙ where A is an amine containing monomer having single unsaturation, B is a vinyl monomer having multiple unsaturations, x=1 to 15, y=1 to 15 and n=10 to 1000. The process comprises the steps of:
a) dissolving an inclusion complex of β-cyclodextrin or derivative thereof with monomer with multiple unsaturation, in a polar solvent in concentration of less than 4 wt %,
b) adding at least one amine containing monomer having single unsaturation and a free radical initiator to the reaction mixture of step (a) and copolymerizing the monomers in the resultant solution mixture and precipitating the resultant product in an organic solvent, followed by washing and drying by known method to obtain the desired water soluble copolymer,
c) crosslinking the copolymer obtained in step (b) by dissolving it in a polar solvent, in the presence of a template molecule to obtain desired crosslinked copolymer,
d) extracting the template molecule from the crosslinked polymer obtained in step (c) in an organic solvent and drying resultant product to obtain desired crosslinked polymer.

In one embodiment the amine containing monomer having single unsaturation is selected from 2-[methyl (acryloyl oxyethyl)] trimethyl ammonium chloride, N-acryloyl-6-amino caproyl hydrochloride, N-acryloyl-5-amino caproyl hydrochloride, 2-amino ethyl acrylate, 2-amino ethyl methacrylate hydrochloride, vinyl amine hydrochloride and allylamine hydrochloride.

In another embodiment the vinyl monomer having multiple unsaturations used is selected from ethylene bis acrylamide, ethylene bis methacrylamide, methylene bis acrylamide, methylene bis methacrylamide, propylene bis acrylamide, propylene bis methacrylamide, butylene bis acrylamide, butylene bis methacrylamide, phenylene bis acrylamide and phenylene bis methacrylamide.

In another embodiment the polar solvent used in step (a) is water.

In another embodiment the organic solvent used in step (b) is an alcohol.

In another embodiment the polar solvent used in step (c) is selected from the group consisting of water, dimethyl formamide and dimethyl sulfoxide.

In another embodiment in step (c) the polymer is crosslinked by either thermal or photochemical polymerization.

In another embodiment the initiator used in step (b) is a water soluble thermal initiator selected from the group consisting of 2, 2′ azo bis [2-amidinopropane] dihydrochloride, potassium persulfate and ammonium persulfate.

In another embodiment the mole ratio of amine functional monomer of the copolymer to template molecule used in step (c) is in the range of 10:1 to 1:2.

In another embodiment the template molecule used in step (c) is selected from taurochenodeoxycholic acid, glycochenodeoxycholic acid, cholic acid, chenodeoxycholic acid, glycocholic acid, taurocbolic acid, deoxycholic acid and lithocholic acid.

Binding Capacity Measurement

Rebinding studies of NaC and NaT were carried out as per the procedure mentioned below. The NaC and NaT solutions were prepared in an aqueous phosphate buffer [pH 7.4] [W. E. Baille, W. O. Huang, M. Nichifor, X. X. Zhu, J. Macromolecular Sci. Part A, 37 [2000] 677]. The concentration of stock solution was 2.6 mg/ml.

In a 50 ml conical flask, 10 mg of polymer was weighed and 4 ml of bile solution was added. Flask was stirred in a circulatory shaking water bath at 37° C. for 3 hrs. The polymer suspension was centrifuged [1000 rpm for 30 min]. In 2 ml of supernatant 200 μl acetic acid was added and diluted to 10 ml with methanol and concentration of cholic acid or taurocholic acid was determined by High Pressure Liquid Chromatography [HPLC].

The ratio of binding capacities of the polymer for NaC to the binding capacities of the polymer for NaT was expressed as selectivity ∝_{NaC/NaT}

The invention is now described below by examples, which are illustrative but do not limit the scope of the invention.

### EXAMPLE 1

### Synthesis of NaC Imprinted Polymers by Simultaneous Polymerization/Crosslinking

In a 100 ml round bottom flask predetermined quantities of MBAM, allylamne hydrochloride and NaC were dissolved in water [Table 1]. In particular for the synthesis of polymer P₁, 2.72 g [2.91×10⁻² Mole] of allylamine hydrochloride, 1.25 g [2.91×10⁻³ Mole] of NaC and 1.50 g [9.70×10⁻³ Mole] of MBAM were dissolved in 40 ml of distilled water. In the round bottom flask 1% by weight of potassium persulfate was added as an initiator and the flask was purged with nitrogen for 30 min. Flask was maintained in a hot water bath at 65° C. for 18 hrs. The template NaC was extracted from the imprinted polymer by Soxhlet extraction for 48 hrs in methanol. Complete extraction was confirmed by verifying that further extraction did not yield any NaC. The polymer was dried and stored at room temperature.

The compositions of polymers, their binding capacities and utilization of active sites is summarized in Table 1

TABLE 1
Rebinding of NaC from imprinted polymers prepared by simultaneous
copolymerization/crosslinking

| | Feed | | | % Found | | | |
|---|---|---|---|---|---|---|---|
| Polymer | C g | M g | T g | C | M | NaC (mg/g) | U (%) |
| P₁ | 1.5 | 2.72 | 1.25 | 72 | 28 | 290 | 33 |
| P₂ | 1.5 | 2.72 | 2.50 | 71 | 29 | 310 | 34 |
| P₃ | 1.5 | 2.72 | 12.56 | 77 | 23 | 345 | 36 |
| P₄ | 1.5 | 2.72 | 25.00 | 72 | 28 | 357 | 40 |
| P₅ | 1.5 | 8.40 | 38.66 | 61 | 39 | 422 | 33 |
| P₆ | 0.75 | 5.91 | 27.21 | 62 | 38 | 435 | 34 |
| P₇ | 0.75 | 8.18 | 37.67 | 56 | 44 | 475 | 33 |

C = Crosslinker MBAM,
M = Monomer allylamine hydrochloride,
T = Template NaC,
U % = % Utilization of functional monomer during rebinding of sodium cholate.

### EXAMPLE 2

### Synthesis of NaT Imprinted Polymers by Simultaneous Polymerization/Crosslinking

In a 100 ml round bottom flask P₈ [Table 2], 5 g [3.0×10⁻² Mole] of 2-amino ethyl methacrylate hydrochloride, 16 g [3.0×10⁻² Mole] of NaT and 2 g [1.02×10⁻² Mole] of EBMA were dissolved in 40 ml of distilled water. In the round bottom flask 1% by weight of potassium persulfate was added as an initiator and the flask was purged with nitrogen for 30 min. Flask was maintained in a hot water bath at 65° C. for 18 hrs. The template NaT was extracted from the imprinted polymer by Soxblet extraction for 48 hrs in methanol. Complete extraction was confirmed by verifying that farther extraction did not yield any NaT. The polymer was dried and stored at room temperature.

The binding capacities and utilization of active sites is summarized in Table 2

TABLE 2
Rebinding of bile salts from imprinted polymers

| | % Found | | | | | |
|---|---|---|---|---|---|---|
| Polymer | C | M | NaT (mg/g) | U (%) | NaC (mg/g) | ^{∝}NaT/NaC |

Imprinted polymer prepared by simultaneous copolymerization/
crosslinking

| | | | | | | |
|---|---|---|---|---|---|---|
| P₈ | 77 | 23 | 358 | 37 | 220 | 1.62 |

Imprinted polymer prepared by sequential copolymerization/
crosslinking

| | | | | | | |
|---|---|---|---|---|---|---|
| P₉ | 78 | 22 | 475 | 71 | 120 | 3.95 |

C = Crosslinker EBMA,
M = Monomer 2 - amino ethyl methacrylate hydrochloride,
U % = Utilization of functional monomer during rebinding of NaT.

### EXAMPLE 3

### Synthesis of NaC Imprinted Polymers by Sequential Polymerization/Crosslinking

Stage 1: Synthesis of Copolymer of MBAM and allylamine hydrochloride

Predetermined quantities of MBAM complex and allylamine hydrochloride monomer were dissolved in water [Table 3]. In particular for the synthesis of polymer P₁₀, 4 g [2.69×10⁻³ Mole] of MBAM complex and 0.754 g [8.07×10⁻³ Mole] of allylamine hydrochloride were dissolved in 120 ml of distilled water. To the round bottom flask 1% by weight of potassium persulfate was added as an initiator and nitrogen was purged for 30 min. Flask was maintained in a hot water bath at 65° C. for 18 hrs. Copolymer was precipitated into methanol and separated by filtration. Polymer was dried and characterized.

Yield: 0.58 g [50%]

¹H NMR [300 MHz D₂O]: 5.44 δ m [2H, CH₂— of MBAM], 5.26 δ m [2H, —CH—CO—], 4.54 δ m [2H, —NH—CH₂—NH—], 3.40 δ s [6H, —CH₃] 3.05 δ s [1H, —CH— of allylamine], 2.27 δ m [1H, —CH—CH—], 1.77 δ d [2H, —CH—CH₂—]

TABLE 3
Synthesis of copolymer of MBAM and allylamine hydrochloride

| | |
|---|---|
| Feed | % Found |

| Polymer | C g | M g | C | M |
|---|---|---|---|---|
| P₁₀ | 4 | 0.75 | 79 | 21 |
| P₁₁ | 4 | 0.75 | 79 | 21 |
| P₁₂ | 4 | 0.75 | 79 | 21 |
| P₁₃ | 4 | 0.75 | 79 | 21 |
| P₁₄ | 4 | 2.32 | 74 | 26 |
| P₁₅ | 4 | 3.20 | 77 | 23 |
| P₁₆ | 4 | 4.50 | 73 | 27 |

C = MBAM complex, M = Monomer allylamine hydrochloride.

Stage 2: Crosslinking of MBAM/allylamine hydrochloride Copolymer in the Presence of Template NaC

Predetermined quantities of copolymer and template were dissolved in water [Table 4]. In particular for the synthesis of polymer P₁₀, 0.768 g of the MBAM/allylamine hydrochloride copolymer [containing 1.15×10⁻³ Moles of allylamine hydrochloride] and 0.050 g [1.15×10⁻⁴ Mole] of NaC as template were dissolved in 2.5 ml of distilled water. In the round bottom flask 1% by weight of potassium persulfate was added as an initiator and nitrogen was purged for 30 min. Flask was maintained in a hot water bath at 65° C. for 18 hrs. The template NaC was extracted from the imprinted polymer by Soxhlet extraction for 48 hrs in methanol. Complete extraction was confirmed by verifying that further extraction did not yield any NaC. The polymer was dried and stored at room temperature. The compositions of polymers, their binding capacities and utilization of active sites is summarized in Table 4 and 5

TABLE 4
Rebinding of NaC from imprinted polymers prepared by sequential
copolymerization/crosslinking

Feed

| Polymer | Copolymer g | T g | NaC mg/g | U % |
|---|---|---|---|---|
| P₁₀ | 0.768 | 0.05 | 325 | 51 |
| P₁₁ | 0.768 | 0.099 | 385 | 60 |
| P₁₂ | 0.768 | 0.61 | 435 | 68 |
| P₁₃ | 0.768 | 0.99 | 475 | 74 |
| P₁₄ | 0.744 | 0.63 | 571 | 70 |
| P₁₅ | 0.844 | 0.71 | 579 | 84 |
| P₁₆ | 0.875 | 0.88 | 587 | 71 |

T = Template NaC, U % = % Utilization of functional monomer in MIP.

TABLE 5
Selectivity studies for NaC imprinted polymers

| Polymer | NaC (mg/g) | NaT (mg/g) | ^{∝}NaC/NaT |
|---|---|---|---|
| P₁ | 290 | 186 | 1.55 |
| P₂ | 310 | 220 | 1.40 |
| P₃ | 345 | 281 | 1.22 |
| P₄ | 357 | 247 | 1.44 |
| P₅ | 422 | 387 | 1.09 |
| P₆ | 435 | 395 | 1.10 |
| P₇ | 475 | 416 | 1.14 |
| P₈ | 325 | 197 | 1.64 |
| P₉ | 385 | 215 | 1.79 |
| P₁₀ | 435 | 185 | 2.35 |
| P₁₁ | 475 | 175 | 2.71 |
| P₁₂ | 571 | 196 | 2.91 |
| P₁₃ | 579 | 187 | 3.09 |
| P₁₄ | 587 | 195 | 3.01 |

### EXAMPLE 4

### Synthesis of NaT Imprinted Polymers by Sequential Polymerization/Crosslinking

Stage 1: Synthesis of Copolymer of EBMA and 2-amino ethyl methacrylate hydrochloride

For synthesis of polymer P₉ [Table 2], 4 g [2.61×10⁻³ Mole] of EBMA complex and 1.303 g (7.83×10⁻³ Mole] of 2-amino ethyl methacrylate hydrochloride were dissolved in 120 ml of distilled water. To a round bottom flask 1% by weight of potassium persulfate was added as an initiator and nitrogen was purged for 30 min. Flask was maintained in a hot water bath at 65° C. for 18 hrs. Copolymer was precipitated into methanol and separated by filtration. Polymer was dried and characterized. Yield: 0.68 g [52%]

¹H NMR [300 MHz D₂O]: 3.44 δ t [2H, —O—CH₂—], 5.44, 5.65 δ t [2H, =CH₂], 1.95 δ q [3H —CH₃ of EBMA], 1.89 δ q [3H, —CH₃], 4.27 δ t [2H, O—CH₂—], 3.44 δ t [2H, —CH₂—NH—] of 2-amino ethyl methacrylate hydrochloride.

Stage 2: Crosslinking of EBMA/2-amino ethyl methacrylate hydrochloride Copolymer in the Presence of Template NaT

For the synthesis of polymer P₉, 0.768 g of the EBMA/2-amino ethyl methacrylate hydrochloride copolymer [containing 1.02×10⁻³ Moles of 2-amino ethyl methacrylate hydrochloride] and 0.548 g [1.02×10⁻³ Mole] of NaT as template were dissolved in 2.5 ml of distilled water. In the round bottom flask 1% by weight of potassium persulfate was added as an initiator and nitrogen was purged for 30 min. Flask was maintained in a hot water bath at 65° C. for 18 hrs. The template NaT was extracted from the imprinted polymer by Soxhlet extraction for 48 hrs in methanol. Complete extraction was confirmed by verifying that further extraction did not yield any NaT. The polymer was dried and stored at room temperature. The binding capacities and utilization of active sites is summarized in Table 2

We claim:

1. A bile acid imprinted crosslinked polymer of the formula [A_{[x]} B_{[y]}]ₙ where, A is an amine containing monomer having single unsaturation, B is a vinyl monomer having multiple unsaturation, x=1 to 15, y=1 to 15 and n=10 to 1000.

2. A bile acid imprinted crosslinked polymer as claimed in claim 1 wherein the amine containing monomer with single unsaturation is selected from the group consisting of 2-[methyl (acryloyl oxyethyl)] trimethyl ammonium chloride, N-acryloyl-6-amino caproyl hydrochloride, N-acryloyl-5-amino caproyl hydrochloride, 2-amino ethyl acrylate, 2-amino ethyl methacrylate hydrochloride, vinyl amine hydrochloride and allylamine hydrochloride.

3. A bile acid imprinted crosslinked polymer as claimed in claim 1 wherein the vinyl monomer with multiple unsaturation is selected from the group consisting of ethylene bis acrylamide, ethylene bis methacrylamide, methylene bis acrylamide, methylene bis methacrylamide, propylene bis acrylamide, propylene bis methacrylamide, butylene bis acrylamide, butylene bis methacrylamide, phenylene bis acrylamide and phenylene bis methacrylamide.

4. A bile acid imprinted crosslinked polymer as claimed in claim 1 wherein the content of amine containing monomer is in the range of 61 to 90 mole percent.

5. A process for the preparation of bile acid imprinted crosslinked polymer of formula [A_{[x]} B_{[y]}]ₙ where A is an amine containing monomer with single unsaturation, B is a vinyl monomer with multiple unsaturations, x=1 to 15, y=1 to 15 and n=10 to 1000, the process comprising the steps of:
a) dissolving an inclusion complex made of β-cyclodextrin or a derivative thereof with monomer having multiple unsaturations, in a polar solvent in concentration of less than 4 wt %, to form a reaction mixture;
b) adding at least one amine containing monomer having single unsaturation and a free radical initiator to the reaction mixture of step (a) to form a solution mixture, and copolymerizing the monomers in the resultant solution mixture and precipitating the resultant product in an organic solvent, followed by washing and drying to obtain a desired water soluble copolymer,
c) crosslinking the copolymer obtained in step (b) by dissolving it in a polar solvent, in the presence of a template molecule to obtain a desired crosslinked copolymer,
d) extracting the template molecule from the crosslinked polymer obtained in step (c) in an organic solvent and drying the resultant product to obtain the desired crosslinked polymer.

6. A process as claimed in claim 5 wherein the amine containing monomer with single unsaturation is selected from the group consisting of 2-[methyl (acryloyl oxyethyl)] trimethyl ammonium chloride, N-acryloyl-6-amino caproyl hydrochloride, N-acryloyl-5-amino caproyl hydrochloride, 2-amino ethyl acrylate, 2-amino ethyl methacrylate hydrochloride, vinyl amine hydrochloride and allylamine hydrochloride.

7. A process as claimed in claim 5 wherein the vinyl monomer with multiple unsaturations is selected from the group consisting of ethylene bis acrylamide, ethylene bis methacrylamide, methylene bis acrylamide, methylene bis methacrylamide, propylene bis acrylamide, propylene bis methacrylamide, butylene bis acrylamide, butylene bis methacrylamide, phenylene bis acrylamide and phenylene bis methacrylamide.

8. A process as claimed in claim 5 wherein the polar solvent used in step (a) is water.

9. A process as claimed in claim 5 wherein the organic solvent used in step (b) is an alcohol.

10. A process as claimed in claim 5 wherein the polar solvent used in step (c) is selected from the group consisting of water, dimethyl formamide and dimethyl sulfoxide.

11. A process as claimed in claim 5 wherein in step (c) the polymer is crosslinked by either thermal or photochemical polymerization.

12. A process as claimed in claim 5 wherein the initiator used in step (b) is a water soluble thermal initiator selected from the group consisting of 2,2′ azo bis [2-amidino propane] dihydrochloride, potassium persulfate and ammonium persulfate.

13. A process as claimed in claim 5 wherein the mole ratio of amine functional monomer of the copolymer to template molecule used in step (c) is in the range of 10:1 to 1:2.

14. A process as claimed in claim 5 wherein the template molecule used in step (c) is selected from the group consisting of taurochenodeoxycholic acid, glycochenodeoxycholic acid, cholic acid, chenodeoxycholic acid, glycocholic acid, taurocholic acid, deoxycholic acid and lithocholic acid.

15. A process as claimed in claim 5 wherein the content of amine containing monomer is in the range of 61 to 90 mole percent.
